(19) United States
(12) Patent Application Publication
(10) Pub. No.: US 2005/0014847 A1
(43) Pub. Date: Jan. 20, 2005

(54) Ambroxol for the treatment of chronic nociceptive pain

(75) Inventors: Wolfram Gaida, Ingelheim am Rhein (DE); Klaus Klinder, Oggelshausen (DE); Thomas Weiser, Biberach (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim International GmbH, Ingelheim (DE)

(21) Appl. No.: 10/888,381

(22) Filed: Jul. 9, 2004

(30) Foreign Application Priority Data
Jul. 16, 2003 (DE) DE 103 32 487

(51) Int. Cl. A61K 31/137

(52) U.S. Cl. 514/650

(57) ABSTRACT

A method of treating chronic nociceptive pain, osteoarthritis, irritable bowel syndrome, fibromyalgia, visceral pain, or rheumatoid arthritis in a patient in need thereof, the method comprising administering to the patient ambroxol or a pharmacologically acceptable salt thereof.

### RELATED APPLICATIONS

This application claims priority to German patent application No. DE 103 32 487.9, filed Jul. 16, 2003, which is hereby incorporated by reference in its entirety.

### FIELD OF THE INVENTION

The invention relates to the use of ambroxol and the pharmacologically acceptable salts thereof for preparing a pharmaceutical composition for the treatment of chronic nociceptive pain.

### BACKGROUND OF THE INVENTION

The active substance ambroxol (trans-4-(2-amino-3,5-dibromobenzylamino)cyclohexanol) is a known local anesthetic, antitussive, and expectorant. In addition, ambroxol's effect as a sodium channel blocker is described in the literature (Society for Neuroscience Abstracts, 2000, Vol. 26, No. 1-2). The potential activity of sodium channel blockers as pain relievers is also known from the prior art (Mao and Chen (2000), Pain 87, 7-17). Known sodium channel blockers, however, are not fundamentally suitable for treating chronic pain as they preferentially inhibit those sodium channels which play a subordinate role in the generation and transmission of noxic signals in sensory neurons, i.e., those which may be inhibited by tetrodoxin, unlike tetrodoxin-resistant neuronal sodium channels (Rush and Elliott (1997), Neuroscience Letters 226, 95-98; Scholz et al. (1998); Journal of Neurophysiology 79, 1746-1754; Song et al. (1997), Journal of Pharmacology and Experimental Therapeutics, 282, 707-714).

Known sodium channel blockers generally act selectively by blocking the sodium channels. In addition, they are not suitable for oral administration in every case and frequently exhibit both cardiovascular and central nervous side effects (Groban (2003), Regional Anesthesia and Pain Medicine 28, 3-11, Webb and Kamali (1998), Pain 76, 357-363).

It is also known from the prior art that calcium channel blockers and AMPA receptor agonists (α-amino-3-hydroxy-5-methyl-4-isoxazolepropionate) exhibit good effects in pain models (Sluka (1998), JPET 287, 232-237; Saegusa, Matsuda, and Tanabe, (2002), Neurosci Res 43, 1-7; Szekely et al. (1997), Europ J Pharmacol 336, 143-154).

The aim of the present invention is to provide an active substance for the treatment of chronic nociceptive pain, particularly osteoarthritis, rheumatoid arthritis, visceral pain, pain caused by tumors, irritable bowel syndrome (IBS), or fibromyalgia, which has no or only insignificant central nervous and cardiovascular side-effects. In addition, the active substance provided in addition to having a potent antinociceptive activity should be suitable for oral administration and hence have good bioavailability.

### DESCRIPTION OF THE INVENTION

Surprisingly, ambroxol shows a very good activity in the treatment of chronic nociceptive pain, particularly osteoarthritis, rheumatoid arthritis, visceral pain, pain caused by tumors and irritable bowel syndrome, based inter alia on blocking the sodium channels. At a pharmacologically effective dose, there are no central nervous or cardiovascular side-effects. Surprisingly, ambroxol also exhibits very good effects as a calcium channel blocker and as an AMPA receptor antagonist, which give rise to an additional potent antinociceptive activity.

The invention therefore relates to the use of ambroxol or one of the pharmacologically acceptable salts thereof for preparing a pharmaceutical composition for the oral treatment of chronic nociceptive pain.

Preferably ambroxol or one of the pharmacologically acceptable salts thereof is used to prepare a pharmaceutical composition for the treatment of osteoarthritis.

Also preferred is the use of ambroxol or one of the pharmacologically acceptable salts thereof for preparing a pharmaceutical composition for the treatment of irritable bowel syndrome (IBS).

It is also preferred to use ambroxol or one of the pharmacologically acceptable salts thereof for preparing a pharmaceutical composition for the treatment of fibromyalgia.

It is particularly preferred to use ambroxol or one of the pharmacologically acceptable salts thereof for preparing a pharmaceutical composition for the treatment of visceral pain.

Particularly preferred is the use of ambroxol or one of the pharmacologically acceptable salts thereof for preparing a pharmaceutical composition for the treatment of rheumatoid arthritis.

It is also particularly preferred to use ambroxol or one of the pharmacologically acceptable salts thereof for preparing a pharmaceutical composition for the treatment of chronic pain caused by tumors.

The invention further relates to the use of a pharmaceutical composition for oral administration containing ambroxol or one of the pharmacologically acceptable salts thereof.

Also preferred is the use of ambroxol as described above, wherein ambroxol is used in a daily dose of from 30 mg to 4000 mg, preferably from 150 mg to 3000 mg, more preferably 350 mg to 2500 mg, and most preferably from 500 mg to 2000 mg.

The invention also relates to a pharmaceutical composition containing ambroxol and one or more active substances selected from the group consisting of analgesics, NSAIDs, arylacetic acid derivatives, arylpropionic acid derivatives, anthranilic acid derivatives, pyrazolone derivatives, oxicams, opioids, anticonvulsants, local anesthetics, antidepressants and glutamate receptor antagonists, preferably salicylic acid derivatives, particularly acetylsalicylic acid, diclofenac, ibuprofen, indometacin, paracetamol, flufenamic acid, mefenamic acid, morphine, pethidine, methadone, fentanyl, buprenorphine, tramadol, gabapentin, pregabalin, carbamazepine, lamotrigin, topiramate, phenyloin, levitiracetam, procaine, lidocaine, mepivacaine, articaine, prilocaine, etidocaine, bupivacaine, ropivacaine, amitryptiline, paroxetine, citalopram, bupropione, duxoletine, ketamine, memantine, 2,3-benzodiazepines, gyki compounds, and quinoxaline-diones.

The invention further relates to the use of ambroxol or one of the pharmacologically acceptable salts thereof in combination with one or more other active substances, selected from the group consisting of analgesics, NSAIDs, arylacetic acid derivatives, arylpropionic acid derivatives, anthranilic acid derivatives, pyrazolone derivatives, oxicams, opioids, anticonvulsants, local anesthetics, antidepressants and glutamate receptor antagonists, preferably salicylic acid derivatives, particularly acetylsalicylic acid, diclofenac, ibuprofen, indometacin, flufenamic acid, mefenamic acid, morphine, pethidine, methadone, fentanyl, buprenorphine, tramadol, gabapentin, pregabalin, carbamazepine, lamotrigin, topiramate, phenyloin, levitiracetam, procaine, lidocaine, mepivacaine, articaine, prilocaine, etidocaine, bupivacaine, ropivacaine, amitryptiline, paroxetine, citalopram, bupropione, duxoletine, ketamine, memantine, 2,3-benzodiazepines, gyki compounds, and quinoxaline-diones.

Ambroxol is most preferably used to treat patients with chronic nociceptive pain combined with other forms of pain, for example, mixed forms of chronic pain, chronic neuropathic pain, or acute pain, preferably chronic neuropathic pain.

The name ambroxol within the scope of the present invention denotes both the base ambroxol, and also the solvates or hydrates thereof, preferably the base ambroxol.

Acids suitable for forming salts of ambroxol are for example hydrochloric acid, hydrobromic acid, sulfuric acid, phosphoric acid, nitric acid, oxalic acid, malonic acid, fumaric acid, maleic acid, tartaric acid, citric acid, ascorbic acid, and methanesulfonic acid, preferably hydrochloric acid.

The effects of ambroxol according to the invention will be illustrated by the Examples that follow. These serve merely to illustrate the invention and are not to be regarded as limiting it.

Ambroxol has an antinociceptive activity which is based inter alia on the blocking of voltage-dependent sodium channels. Unlike the sodium channel blockers described which are in clinical use, ambroxol preferentially inhibits tetrodotoxin-resistant sodium channels in nociceptive C-fiber neurons. Their particular relevance for inflammatory and chronic pain has been demonstrated in vivo (Waxman et al. (1999) Proc Nat Acad Sci USA 96, 7635-7639; Khasar et al. (1998), Neurosci Lett 256, 17-20, (J M A Laird et al. (2001), BJP 134, 1742-1748).

In neuron cultures from the posterior root ganglia of adult rats, tetrodotoxin-resistant sodium channels were half-maximally inhibited by 35 μM ambroxol. Tetrodotoxin-sensitive currents were inhibited much less powerfully by this concentration, the IC₅₀ here being more than 100 μM.

Voltage-dependent calcium channels play an important role in neurotransmission. It has been found, surprisingly, that ambroxol also blocks voltage-dependent calcium channels in neuron cultures from rats in concentrations of 10 μM to 1000 μM. Neurons were dissected from posterior root ganglia of adult rats and placed in short-term culture. The cells were investigated electrophysiologically by the Patch-Clamp method (voltage terminal), and the flow of current through voltage-dependent calcium channels was measured after electrical stimulation (voltage jumps from −80 mV to 0 mV holding potential for 50 ms) in the presence and absence of ambroxol.

Ionotropic glutamate receptors of the AMPA sub-type are also essential for the excitatory neurotransmission. In HEK 293 cells which express heterologously human GluR1/2 receptors, ambroxol surprisingly inhibits glutamate-induced membrane currents in concentrations ranging from 30-1000 μM. HEK 293 cells which expressed functionally recombinant human GluR1/2 receptors were electrophysiologically investigated by the Patch-Clamp method (voltage terminal). The administration of 1 mM glutamate (for 1 s at a holding potential of −80 mV) induced membrane currents which were inhibited by the joint administration of ambroxol.

Ambroxol may be used on its own or in combination with other pharmacologically active substances. Suitable preparations include for example tablets, capsules, suppositories, solutions, elixirs, emulsions, or dispersible powders, preferably tablets. Suitable tablets may be obtained, for example, by mixing the active substance(s) with known excipients, for example inert diluents such as calcium carbonate, calcium phosphate, or lactose, disintegrants such as corn starch or alginic acid, binders such as starch or gelatine, lubricants such as magnesium stearate or talc and/or agents for delaying release, such as carboxymethyl cellulose, cellulose acetate phthalate or polyvinyl acetate. The tablets may also comprise several layers.

Coated tablets may be prepared accordingly by coating cores produced analogously to the tablets with substances normally used for tablet coatings, for example, collidone or shellac, gum arabic, talc, titanium dioxide, or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations thereof according to the invention may additionally contain a sweetener such as saccharine, cyclamate, glycerol, or sugar and a flavor enhancer, e.g., a flavoring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection are prepared in the usual way, e.g., with the addition of preservatives such as p-hydroxybenzoates, or stabilizers such as alkali metal salts of ethylenediamine tetraacetic acid, and transferred into injection vials or ampoules.

Capsules containing one or more active substances or combinations of active substances may, for example, be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

A therapeutically effective daily dose is 30 mg to 4000 mg, preferably 150 mg to 3000 mg, more preferably 350 mg to 2500 mg, and most preferably 500 mg to 2000 mg of ambroxol, in adults.

The Examples which follow illustrate the present invention without restricting its scope.

Examples of Pharmaceutical Formulations

| A) | Tablets | per tablet |
|---|---|---|
| | ambroxol | 800 mg |
| | lactose | 140 mg |
| | maize starch | 240 mg |
| | polyvinylpyrrolidone | 20 mg |
| | magnesium stearate | 10 mg |

Ambroxol, lactose, and some of the maize starch are mixed together. The mixture is screened, then moistened with a solution of polyvinylpyrrolidone in water, kneaded, wet-granulated and dried. The granules, the remaining maize starch, and the magnesium stearate are screened and mixed together. The mixture is compressed to produce tablets of suitable shape and size.

| B) | Tablets | per tablet |
|---|---|---|
| | ambroxol | 800 mg |
| | maize starch | 190 mg |
| | lactose | 55 mg |
| | microcrystalline cellulose | 35 mg |
| | polyvinylpyrrolidone | 20 mg |
| | sodium-carboxymethyl starch | 30 mg |
| | magnesium stearate | 10 mg |

Ambroxol, some of the corn starch, lactose, microcrystalline cellulose, and polyvinylpyrrolidone are mixed together, the mixture is screened and worked with the remaining corn starch and water to form a granulate which is dried and screened. The sodium-carboxymethyl starch and the magnesium stearate are added and mixed in and the mixture is compressed to form tablets of a suitable size.

| C) | Coated Tablets | per coated tablet |
|---|---|---|
| | ambroxol | 500 mg |
| | maize starch | 45 mg |
| | lactose | 30 mg |
| | polyvinylpyrrolidone | 5 mg |
| | magnesium stearate | 5 mg |

Ambroxol, maize starch, lactose and polyvinylpyrrolidone are thoroughly mixed and moistened with water. The moist mass is pushed through a screen with a 1 mm mesh size, dried at about 45° C. and the granules are then passed through the same screen. After the magnesium stearate has been mixed in, convex tablet cores with a diameter of 11 mm are compressed in a tablet-making machine. The tablet cores thus produced are coated in known manner with a covering consisting essentially of sugar and talc. The finished coated tablets are polished with wax.

| D) | Capsules | per capsule |
|---|---|---|
| | ambroxol | 250 mg |
| | maize starch | 268.5 mg |
| | magnesium stearate | 1.5 mg |

Ambroxol and maize starch are mixed and moistened with water. The moist mass is screened and dried. The dry granules are screened and mixed with magnesium stearate. The finished mixture is packed into size 1 hard gelatine capsules.

| E) | Parenteral Solution | |
|---|---|---|
| | ambroxol | 500 mg |
| | citric acid monohydrate | 100 mg |
| | sodium hydroxide | 35 mg |
| | mannitol | 1500 mg |
| | water for inj. | 50 mL |

The ambroxol is dissolved in water at its own pH or optionally at pH 5.5 to 6.5 and mannitol is added to make it isotonic. The solution obtained is filtered free from pyrogens and the filtrate is transferred under aseptic conditions into injection vials which are then sealed with rubber stoppers and autoclaved.

| F) | Suppositories | |
|---|---|---|
| | ambroxol | 450 mg |
| | solid fat | 1650 mg |

The hard fat is melted. At 40° C. ambroxol is homogeneously dispersed therein. The mixture is cooled to 38° C. and poured into slightly chilled suppository moulds.

| | |
|---|---|
| G) | Oral Solution |

| | | |
|---|---|---|
| ambroxol | 150 | mg |
| hydroxyethylcellulose | 50 | mg |
| sorbic acid | 5 | mg |
| sorbitol (70%) | 600 | mg |
| glycerol | 200 | mg |
| flavoring | 15 | mg |
| water | to 10 | mL |

Distilled water is heated to 70° C. Hydroxyethylcellulose is dissolved therein with stirring. After the addition of sorbitol solution and glycerol the mixture is cooled to ambient temperature. At ambient temperature, sorbic acid, flavoring, and ambroxol are added. To eliminate air from the suspension it is evacuated with stirring.

| | |
|---|---|
| H) | Ointment |
| | Composition g/100 g ointment |

| ambroxol | 20 | g |
|---|---|---|
| sodium disulfite | 0.1 | g |
| cetyl alcohol | 10 | g |
| stearyl alcohol | 10 | g |
| white Vaseline | 5 | g |
| perfume oil | q.s. | |
| distilled water | to 100 | g |

The ingredients are processed in the usual way to produce an ointment.

1. A method of treating chronic nociceptive pain, osteoarthritis, irritable bowel syndrome, fibromyalgia, visceral pain, or rheumatoid arthritis in a patient in need thereof, the method comprising administering to the patient ambroxol or a pharmacologically acceptable salt thereof.

2. The method of treating chronic nociceptive pain in a patient in need thereof, the method comprising administering to the patient ambroxol or a pharmacologically acceptable salt thereof.

3. A method of treating osteoarthritis in a patient in need thereof, the method comprising administering to the patient ambroxol or a pharmacologically acceptable salt thereof.

4. A method of treating irritable bowel syndrome in a patient in need thereof, the method comprising administering to the patient ambroxol or a pharmacologically acceptable salt thereof.

5. A method of treating fibromyalgia in a patient in need thereof, the method comprising administering to the patient ambroxol or a pharmacologically acceptable salt thereof.

6. A method of treating visceral pain in a patient in need thereof, the method comprising administering to the patient ambroxol or a pharmacologically acceptable salt thereof.

7. A method of treating rheumatoid arthritis in a patient in need thereof, the method comprising administering to the patient ambroxol or a pharmacologically acceptable salt thereof.

8. The method according to one of claims 1 to 7, wherein the ambroxol or a pharmacologically acceptable salt thereof is administered orally to the patient.

9. The method according to one of claims 1 to 7, wherein the ambroxol or a pharmacologically acceptable salt thereof is administered to the patient in a daily dose of 30 mg to 4000 mg.

10. The method according to claim 8, wherein the ambroxol or a pharmacologically acceptable salt thereof is administered to the patient in a daily dose of 30 mg to 4000 mg.

11. The method according to one of claims 1 to 7, wherein one or more additional active substances selected from analgesics, anticonvulsants, NSAIDs, arylacetic acid derivatives, anthranilic acid derivatives, opioids, anticonvulsants, local anesthetics, antidepressants, and glutamate receptor antagonists are administered to the patient before, after, or simultaneously with the ambroxol or a pharmacologically acceptable salt thereof.

12. A pharmaceutical composition comprising:
(a) ambroxol or a pharmacologically acceptable salt thereof; and
(b) one or more additional active substances selected from analgesics, anticonvulsants, NSAIDs, arylacetic acid derivatives, anthranilic acid derivatives, opioids, anticonvulsants, local anesthetics, antidepressants, and glutamate receptor antagonists.
